(19) United States
(12) Patent Application Publication
(10) Pub. No.: US 2010/0204615 A1
(43) Pub. Date: Aug. 12, 2010

(54) METHOD AND SYSTEM FOR ASSESSING ATHLETIC PERFORMANCE

(75) Inventors: Andrew Kyle, Victoria (CA); Jeffrey Compton, Victoria (CA); Jagmeet Virk, Victoria (CA); Simon Tipler, Victoria (CA); Mathew Petterson, Victoria (CA)

Correspondence Address: OYEN, WIGGS, GREEN & MUTALA LLP;480 - THE STATION, 601 WEST CORDOVA STREET, VANCOUVER, BC, V6B 1G1, CA

(73) Assignee: 6TH DIMENSION DEVICES INC., Victoria, BC (CA)

(21) Appl. No.: 12/161,328

(22) PCT Filed: Jan. 19, 2007
(86) PCT No.: PCT/CA2007/000087

§ 371 Date: Apr. 23, 2010

Related U.S. Application Data
(60) Provisional application No. 60/760,380, filed on Jan. 20, 2006.

(51) Int. Cl. A61B 5/11

(52) U.S. Cl. 600/595

(57) ABSTRACT

A system for assessing athletic performance comprises a mounting device wearable by an athlete, a sensing device attachable to the mounting device, and a base unit. The sensing device comprises acceleration sensors for measuring acceleration data during an athletic test to produce at least three acceleration signals, rotation sensors for measuring rotation data during the athletic test to produce at least three rotation signals, signal conditioning hardware for conditioning the acceleration and rotation signals and sampling the acceleration and rotation signals at a sampling rate to produce acceleration and rotation data, and, a wireless communication device for transmitting the data. The base unit comprises a wireless communication device for receiving the data, a feature extractor for extracting features relating to athletic performance from the data based on a plurality of expected events of the athletic test, and, an output device for outputting the extracted features.

### CROSS REFERENCE TO RELATED APPLICATION

This application claims the benefit of the filing date of U.S. patent application No. 60/760,380 filed on 20 Jan. 2006 and entitled “METHOD AND SYSTEM FOR ASSESSING ATHLETIC PERFORMANCE”.

### TECHNICAL FIELD

This invention relates to methods and systems for assessing athletic performance. In particular, this invention relates to methods and systems for collecting acceleration and rotation data and extracting features which relate to athletic performance therefrom.

### BACKGROUND

In high performance sport, it is common for an athlete to work closely with a trainer. The role of the trainer is to assist the athlete in physical conditioning. The trainer often measures the physical performance of the athlete and recommends training regimes based on this information.

There are a number of prior art devices which may be used to monitor the motion of a person or other subject. For example, U.S. Pat. No. 5,955,667 to Fyfe discloses a device comprising a pair of accelerometers and a tilt sensor mounted in fixed relation to a datum defining plane such as the sole of a shoe. The device disclosed by Fyfe maybe used for extracting kinematic variables including linear and rotational acceleration, velocity and position.

U.S. Pat. No. 6,305,221 to Hutchings discloses a device that measures the distance traveled, speed, and height jumped of a person while running or walking. The device comprises accelerometers and rotational sensors positioned in the sole of a shoe along with an electronic circuit that performs mathematical calculations to determine the distance and height of each step. A transmitter sends the distance and height information to a central receiving unit which comprises a microprocessor which outputs the distance traveled, speed, or height jumped of the runner or walker to a display.

There exists a need for methods and systems which provide more information about athletic performance.

### SUMMARY

The following embodiments and aspects thereof are described and illustrated in conjunction with systems, tools and methods which are meant to be exemplary and illustrative, not limiting in scope. In various embodiments, one or more of the above-described problems have been reduced or eliminated, while other embodiments are directed to other improvements.

One aspect of the invention provides a system for assessing athletic performance comprises a mounting device wearable by an athlete, a sensing device attachable to the mounting device, and a base unit. The sensing device comprises acceleration sensors for measuring acceleration data during an athletic test to produce at least three acceleration signals, rotation sensors for measuring rotation data during the athletic test to produce at least three rotation signals, signal conditioning hardware for conditioning the acceleration and rotation signals and sampling the acceleration and rotation signals at a sampling rate to produce acceleration and rotation data, and, a wireless communication device for transmitting the acceleration and rotation data. The base unit comprises a wireless communication device for receiving the acceleration and rotation data, a feature extractor for extracting features relating to athletic performance from the data based on a plurality of expected events of the athletic test, and, an output device for outputting the extracted features.

Another aspect of the invention provides a method for assessing athletic performance of a living subject. The method comprises providing at least three acceleration sensors on the subject configured to measure acceleration along three local axes, providing at least three rotation sensors on the subject configured to measure rotation about the three local axes, monitoring the acceleration sensors and the rotation sensors to produce acceleration data and rotation data, determining an orientation of the three local axes based on the measured rotation data, applying a rotation function to the measured acceleration data based on the determined orientation of the three local axes to generate corrected acceleration data along three global axes, receiving a test identification specifying a plurality of expected events, extracting features relating to athletic performance of the subject by detecting events corresponding to the expected events in the corrected acceleration data, and, outputting the extracted features.

In addition to the exemplary aspects and embodiments described above, further aspects and embodiments will become apparent by reference to the drawings and by study of the following detailed descriptions.

### BRIEF DESCRIPTION OF DRAWINGS

Exemplary embodiments are illustrated in referenced figures of the drawings. It is intended that the embodiments and figures disclosed herein are to be considered illustrative rather than restrictive.

In drawings which illustrate non-limiting embodiments of the invention:

FIG. 1 shows a system for assessing athletic performance according to one embodiment of the invention;

FIG. 2 shows basic elements of a sensing device and a base unit according to one embodiment of the invention;

FIG. 3 shows a sensing device according to another embodiment of the invention;

FIG. 4 shows a sensing device according to another embodiment of the invention;

FIG. 5 shows a base unit according to another embodiment of the invention;

FIG. 6 shows a system for assessing athletic performance according to another embodiment of the invention;

FIG. 7 is a flowchart illustrating steps in a method according to one embodiment of the invention;

FIGS. 8A-E are graphical representations of example acceleration data from a jump test as it is processed by a method according to one embodiment of the invention;

FIG. 8F is a graphical representation of velocity data obtained from the example acceleration data of FIG. 8E;

FIG. 9 is a flowchart illustrating steps in a method of extracting features from acceleration data according to one embodiment of the invention;

FIG. 10 shows features extracted from the example acceleration and velocity data of FIGS. 8E and 8F by a method according to one embodiment of the invention;

FIG. 11 shows example acceleration and rotation data from a running test;

FIG. 12 is a flowchart illustrating steps in a method of assessing athletic performance according to another embodiment of the invention;

FIG. 13 shows an example input/output device according to one embodiment of the invention; and

FIG. 14 shows an example feature extractor according to one embodiment of the invention.

### DESCRIPTION

Throughout the following description specific details are set forth in order to provide a more thorough understanding to persons skilled in the art. However, well known elements may not have been shown or described in detail to avoid unnecessarily obscuring the disclosure. Accordingly, the description and drawings are to be regarded in an illustrative, rather than a restrictive, sense.

The invention provides systems and methods for assessing athletic performance. Some embodiments provide a system for collecting data relating to movement of a subject such as, for example an athlete. The system may collect data generated during a test period when the athlete performs a predetermined action or series of actions, and may extract features relating to athletic performance from the collected data.

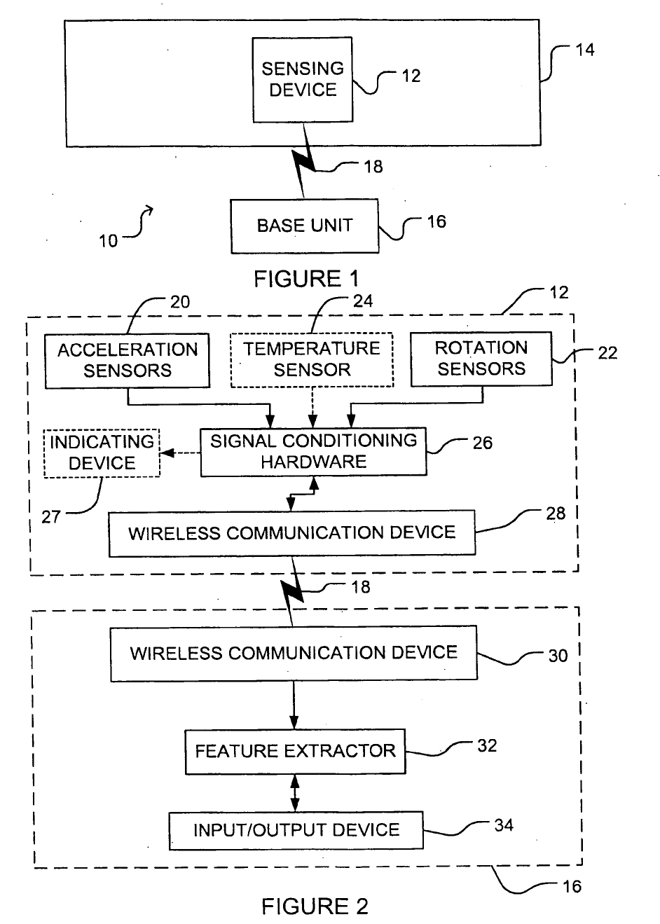

FIG. 1 illustrates a system 10 according to one embodiment of the invention. System 10 comprises a sensing device 12 attachable to a mounting device 14. Mounting device 14 may comprise, for example, a belt, strap or the like which may be worn by an athlete. When in use by an athlete, mounting device 14 may hold sensing device 12 at the small of the athlete's back, since this position is near the athlete's centre of mass and does not impede many athletic activities. However, sensing device 12 may be positioned at another location on the athlete's torso.

Sensing device 12 communicates with a base unit 16 by means of a wireless communication link 18. Base unit 16 may comprise, for example, a personal digital assistant (PDA), a computer, or any other electronic device with suitable data processing capabilities and a communication link.

In operation, an athlete mounts sensing device 12 on his or her body by means of mounting device 14 and performs an action or series of actions (referred to herein as a “test”) designed to assess athletic performance. Sensing device 12 records data during the test, and provides the recorded data to base unit 16. Base unit 16 is also provided with a user-selected identification of the test to be performed by a user such as a trainer, coach, or in some embodiments the athlete who performs the test. Base unit 16 processes the data received from sensing device 12 based on the user-selected test identification to extract features relating to athletic performance. In some embodiments, some data processing is also done by sensing device 12. Base unit 16 provides the extracted features to the user by means of an output device, as discussed further below.

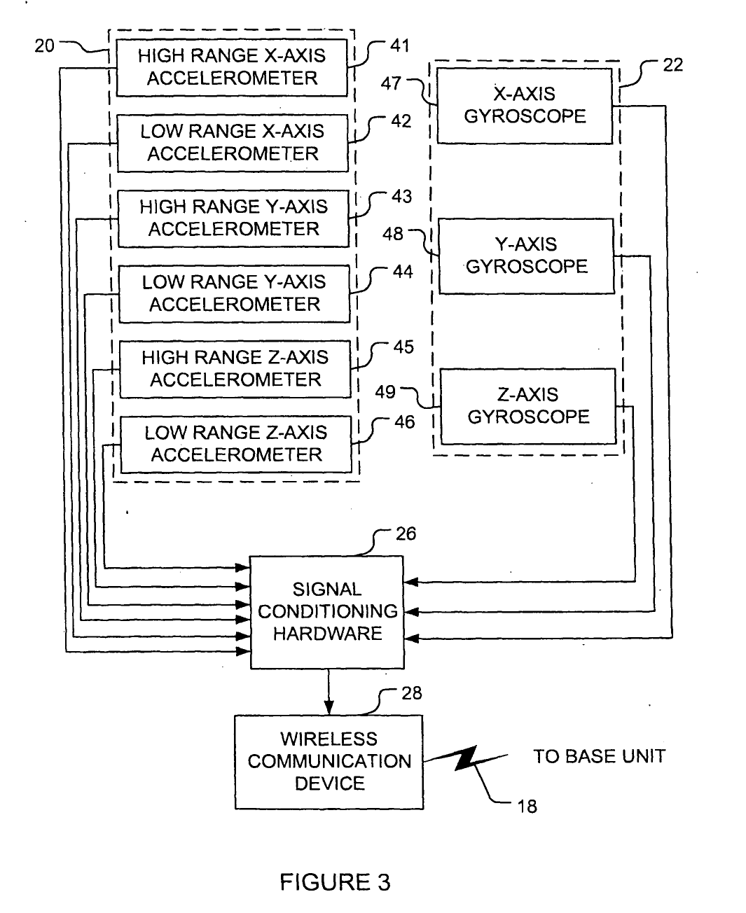

FIG. 2 schematically depicts components of sensing device 12 and base unit 16 according to one embodiment of the invention. Sensing device 12 comprises a plurality of acceleration sensors 20 and a plurality of rotation sensors 22. Acceleration sensors 20 are configured to measure acceleration along each of three local axes and produce at least three acceleration signals which contain acceleration data. Rotation sensors 22 are configured to measure rotation around each of three local axes and produce at least three rotation signals which contain rotation data. The three local axes are referred to herein as the X-axis, Y-axis and Z-axis.

If acceleration sensors 20 and/or rotation sensors 22 are sensitive to temperature, sensing device may optionally comprise at least one temperature sensor 24 (indicated in dotted lines in FIG. 2). Temperature sensor 24 is configured to measure the temperature of acceleration sensors 20 and/or rotation sensors 22 and produce at least one temperature signal which may be used to compensate for variations in the outputs of sensors 20 and/or 22 which may result from changes in temperature.

Sensing device 12 also comprises signal conditioning hardware 26 connected to acceleration sensors 20, rotation sensors 22 and temperature sensors 24 (if applicable). Acceleration sensors 20, rotation sensors 22 and temperature sensors 24 may be analog or digital sensors. If analog sensors are used, signal conditioning hardware may comprise an analog to digital converter (ADC). Signal conditioning hardware 26 is configured to sample the signals from acceleration sensors 20, rotation sensors 22 and temperature sensors 24 at a sampling rate suitable for the test to be performed. The sampling rate may be as low as 50 Hz, but a higher sampling rate may be desirable in some applications. In some embodiments, the sampling rate may be in excess of 100 Hz, for example approximately 400 Hz. Signal conditioning hardware 26 may also comprise, for example, low pass filters for removing high frequency shocks from the signals.

Signal conditioning hardware 26 is connected to provide data from the acceleration, rotation and temperature signals (if applicable) to a wireless communication device 28. Wireless communication device 28 is configured to transmit the data to a compatible wireless communication device 30 associated with base unit 16. Wireless communication devices 28 and 30 may each comprise, for example, a radio frequency (RF) module having a line-of-sight range of one kilometer.

Sensing device 12 may also optionally comprise an indicating device 27 connected to sensor conditioning hardware 26. Indicating device 27 may be operated by sensor conditioning hardware 26 to provide the athlete with a start signal directing the athlete to begin a test. The start signal may comprise, for example, an audible signal, a visual signal, an electrical signal (i.e., a mild shock), or a vibration signal. Sensor conditioning hardware 26 may cause indicating device 27 to provide the start signal in response to a command received from base unit 16 by means of wireless communication devices 28 and 30.

In addition to wireless communication device 30, base unit 16 comprises a feature extractor 32 and an input/output device 34. Feature extractor 32 may comprise, for example, a signal processor coupled to a memory. Input/output device 34 may comprise, for example, a touch-sensitive display, a keyboard and monitor, or the like.

Feature extractor 32 is connected to receive the acceleration, rotation and (if applicable) temperature data from wireless communication device 30. Feature extractor 32 processes the data received from wireless communication device 30 during an athletic test to extract features related to athletic performance. Feature extractor 32 may be programmed with a plurality of expected events for each of a plurality of predetermined tests. A user may select one of the predetermined tests using input/output device 34. Feature extractor 32 may use the expected events for the test identified by the user to extract features related to athletic performance from the data received from sensing device 12. A user may also input provide feature extractor 32 with the athlete's mass using input/output device 34. Feature extractor 32 may use the athlete's mass for extracting features relating to force or power. The features extracted by feature extractor 32 may be provided to a user, the athlete, and/or a data storage medium by means of input/output device 34.

It is to be understood that each of sensing device 12 and base unit 16 also comprise a suitable power source for providing electrical power to the components thereof. The power sources have not been shown to avoid cluttering the drawings.

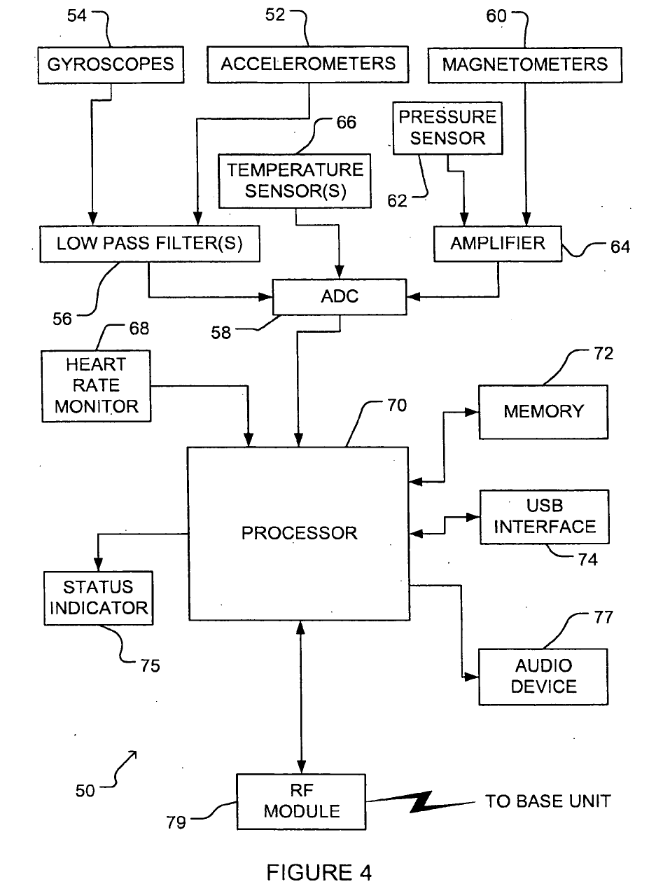

FIG. 3 shows a possible configuration of sensing device 12 according to one embodiment of the invention. In the FIG. 3 embodiment, acceleration sensors 20 comprise six accelerometers 41-46 and rotation sensors 22 comprise three gyroscopes 47-49. Each of the X-, Y- and Z-axes has two acceleration sensors and one rotation sensor associated therewith. Accelerometers 41 and 42 measure acceleration along the X-axis Accelerometers 43 and 44 measure acceleration along the Y-axis. Accelerometers 45 and 46 measure acceleration along the Z-axis. Gyroscope 47 measures rotation about the X-axis. Gyroscope 48 measures rotation about the Y-axis. Gyroscope 49 measures rotation about the Z-axis.

Accelerometers 41, 43 and 45 each have range that is relatively high in comparison to accelerometers 42, 44 and 46 and a sensitivity that is relatively low in comparison to accelerometers 42, 44 and 46. For example, the range of accelerometers 41, 43 and 45 may be 5 g or more (where g represents the acceleration due to gravity at the earth's surface, roughly 9.8 m/s²) and the sensitivity of accelerometers 41, 43 and 45 may be approximately 192 mV/g and the range and sensitivity of accelerometers 42, 44 and 46 may be up to 2 g and approximately 700 mV/g, respectively, although it is to be understood that accelerometers having different ranges and sensitivities may be used. The use of both high range and high sensitivity accelerometers for each local axis allows sensing device 12 to measure large accelerations and changes in acceleration while maintaining the ability to accurately monitor smaller accelerations.

Gyroscopes 47-49 may each comprise a micro-electro-mechanical system (MEMS) configured to measure a rate of rotation about the associated axis. Each of gyroscopes 47-49 may have, for example, a range of 600°/s and a sensitivity of approximately 5 mV/°/s. Gyroscopes 47-49 could each comprise a separate element, or could be combined in a single chip. Alternatively, additional accelerometers could be used instead of gyroscopes 47-49, since rotational information may be provided by two accelerometers positioned to measure acceleration along two spaced apart non-perpendicular axes by using solid body rotation techniques known in the art.

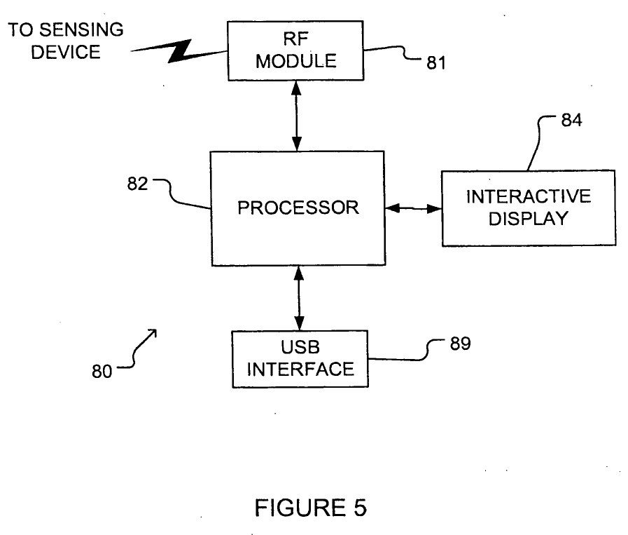

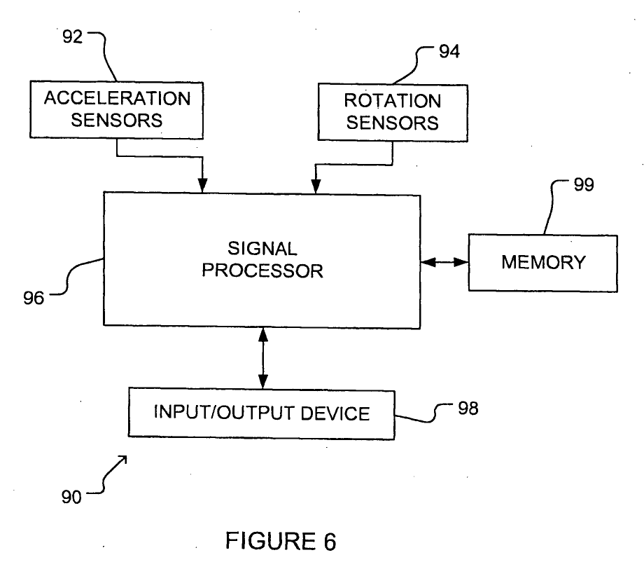

FIGS. 4 and 5 respectively show a sensing device 50 and a base unit 80 of a system for assessing athletic performance according to another embodiment of the invention. The embodiment of FIGS. 4 and 5 is shown for illustrative purposes, and includes a number of features which are not required for the basic functioning of the system, but which may be desirable in some applications.

Sensing device 50 comprises a plurality of accelerometers 52 for measuring acceleration data along three axes to produce at least three acceleration signals and a plurality of gyroscopes 54 for measuring rotation data about three axes to produce at least three rotation signals. The signals from accelerometers 52 and gyroscopes 54 are passed through a low pass filter array 56 in order to remove high frequency noise from the signals. Low pass filter array 56 may comprise, for example, second order operational amplifier-based active filters having a cut off frequency of approximately 100 Hz.

In the FIG. 4 embodiment, accelerometers 52 and gyroscopes 54 produce analog signals. After the acceleration and rotation signals are passed through low pass filter array 56, they are converted to digital signals by an analog to digital converter (ADC) 58. The digital signals from ADC 58 are provided to a processor 70. ADC 58 preferably has an internal clock and is configured to sample analog signals at a suitable sampling rate. The sampling rate of ADC 58 may be, for example, approximately 400 Hz. It is to be understood that ADC 58 is not required in embodiments where digital sensors are used instead of analog sensors. Alternatively, sensing device 50 could provide analog signals to base station 80, in which case ADC 58 may instead be located in base station 80.

Sensing device 50 may also comprise a plurality of magnetometers 60 for measuring the earth's magnetic field in order to produce a magnetic heading signal. Magnetometers 60 may comprise, for example, at least three magnetometers. The magnetic heading signal from magnetometers 60 may be used periodically to verify the orientation of device 50 to compensate for drift which may be caused by accumulation of errors in the rotation signals from gyroscopes 54 as the rotation signals are integrated. Magnetometers 60 may each have, for example, a range of 6 gauss and a sensitivity of approximately 5 mV/gauss. Alternatively, other means for compensating for drift may be used instead of magnetometers 60, such as a gravitometer or a global positioning system (GPS).

Sensing device 50 may also comprise a pressure sensor 62. Pressure sensor 62 measures barometric pressure to produce a pressure signal which may indicate a change in altitude. Pressure sensor 62 may have; for example, a range of 105 kPa and a sensitivity of approximately 20 mV/kPa.

The signals from magnetometer 58 and pressure sensor 60 are also analog signals in the FIG. 4 embodiment. The analog magnetic heading and pressure signals may be passed through an amplifier 64 before being provided to ADC 58. Amplifier 64 may have, for example, a gain of 200 to improve the readability of the magnetic heading and pressure signals by ADC 58.

Sensing device 50 may also comprise at least one temperature sensor 66. Temperature sensor 66 is configured to measure the temperature of any of accelerometers 52, gyroscopes 54, magnetometer 60, and pressure sensor 62 which are temperature sensitive and provide a temperature signal to ADC 58. A single temperature sensor 66 may be positioned in a position which is in a similar thermal environment to the other sensors of sensing device 50, or multiple temperature sensors 66 may be provided, with one positioned near each temperature sensitive sensor.

Sensing device 50 may also comprise a heart rate monitor 68. In the FIG. 4 embodiment, heart rate monitor 68 produces a digital heat rate signal which is provided directly to processor 70.

Processor 70 receives digital acceleration, rotation and optionally other signals and controls the collection of acceleration, rotation and other data over a test period. Processor 70 provides the data to at least one of a memory 72, a USB interface 74 and a RF module 79. Memory 72 may be used to store data from a plurality of tests so that an athlete or trainer may compare results from different tests to track the athlete's progress. USB interface 74 allows processor 70 to be connected to exchange data with other computerized systems. RF module 79 allows processor 70 to communicate with base station 80 (see FIG. 5).

Processor 70 may also control the operation of a status indicator 75. Status indicator 75 may comprise, for example, one or more LEDs which may be selectively illuminated by processor 70 to indicate the status of sensing device 50.

Processor 70 may also control the operation of an audio device 77. Audio device 77 may be used to inform the test subject of the beginning of a test. Processor 70 may receive instructions to initiate a test from another processor 82 in base unit 80 by means of RF modules 79 and 81 (see FIG. 5).

As shown in FIG. 5, base unit 80 comprises an interactive display 84 connected to processor 82. Interactive display 84 may be controlled by software running on processor 82. Interactive display 84 may be used by a user to initiate a test. Interactive display 84 may provide information about the test to a user. Processor 82 may also optionally be connected to a USB interface 89 to allow processor 82 to exchange data with other computerized systems.

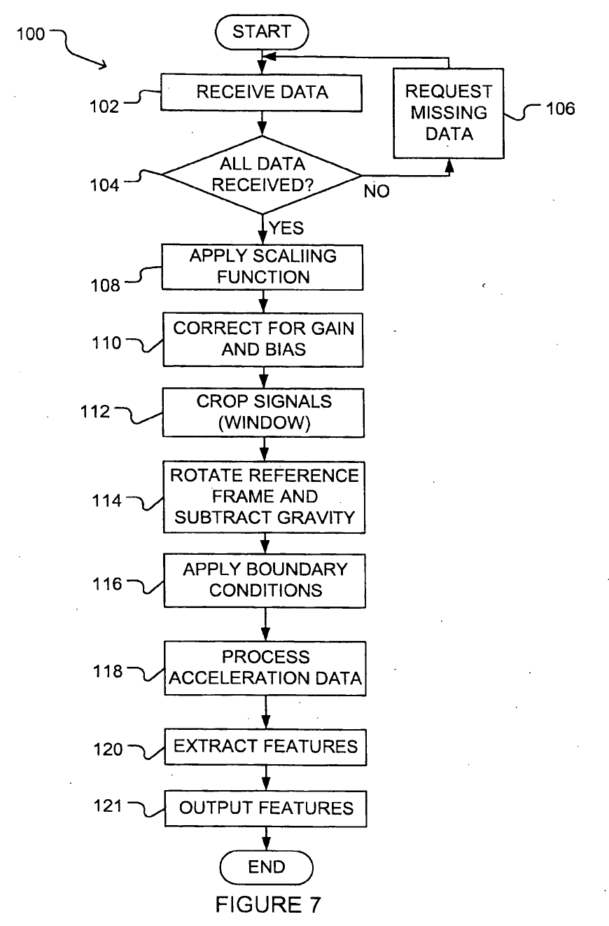

FIG. 6 shows a system 90 for assessing athletic performance according to another embodiment of the invention. System 90 comprises a plurality of acceleration sensors 92 and a plurality of rotation sensors 94 connected to a signal processor 96. Signal processor 96 collects acceleration and rotation data from acceleration and rotation sensors 92 and 94. Signal processor 96 extracts features relating to athletic performance from the acceleration and rotation data and provides the extracted features to an input/output device 98. Input/output device 98 may comprise, for example, a wireless communication device which communicates with a display.

System 90 may also comprise a memory 99. Signal processor 96 may store the extracted features in memory 99. Memory may also contain data relating to a plurality of predetermined expected test events. The expected test events may be used by signal processor 96 in extracting the features relating to athletic performance.

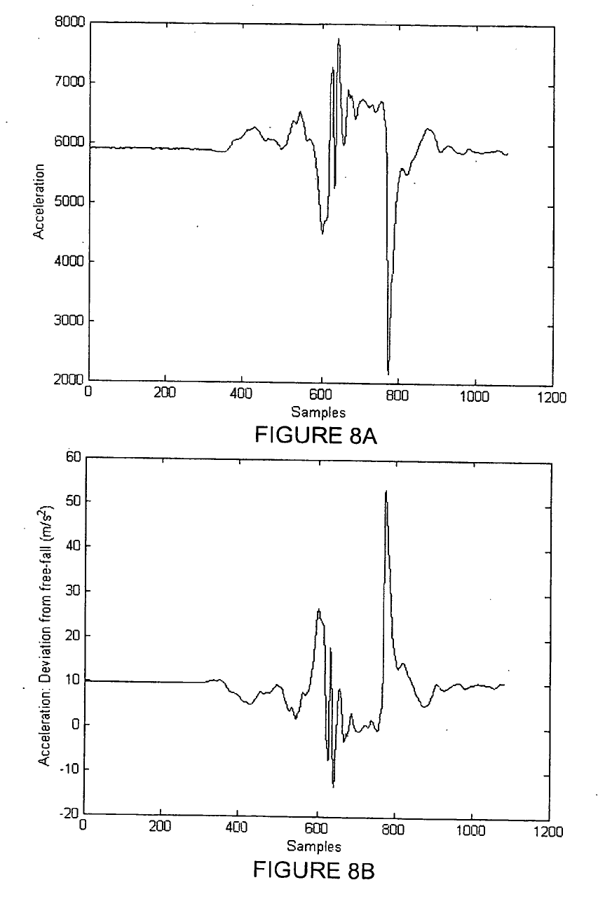

FIG. 7 is a flowchart illustrating a method 100 for assessing athletic performance according to one embodiment of the invention. Method 100 may be carried out by a processor such as, for example, feature extractor 32 in the embodiment of FIGS. 1 and 2, processor 70 or 82 in the embodiment of FIGS. 4 and 5, or signal processor 96 in the embodiment of FIG. 6. Method 100 may be embodied in software stored in a memory accessible to the processor.

At block 102 the processor receives acceleration data and rotation data collected over a test period during which an athlete performs a test. The test period may be initiated by the processor by providing the athlete with an indication that data is being collected. The indication may be provided, for example, by means of input/output device 34 in the FIG. 2 embodiment, or by means of audio device 77 in the embodiment of FIGS. 4 and 5. During the test period, the athlete performs a test comprising a predetermined action or series of actions designed to assess athletic performance. The test period may end after a predetermined amount of time, after the processor detects that the athlete has completed the predetermined action or series of actions, or may be ended manually.

The test may comprise, for example, a single jump test, a multiple jump test, a running test, a sprinting test, a gait analysis test, an agility test, a balance test, a running vertical jump test, a triple jump test, a long jump test, a high jump test, a pole vault test, a reaction time test, a T-test, a zig-zag test, or any other action or series of actions designed to test athletic performance. For each type of test, the processor may be provided with an expected event or set of events which should be represented by the data collected during the test period.

In some embodiments, the sensing device or base unit may provide the athlete with instructions for the test. For example, for a jump test, the sensing device or base unit may instruct the athlete to remain motionless until they hear a tone, then jump straight up. In some embodiments, the athlete is instructed to remain stationary for a first stationary period immediately before the test and/or a second stationary period immediately after the test. The amount of time the athlete remains stationary before and after the test may be, for example about 0.2 seconds. Data collected during the stationary period(s) may be used to provide a baseline reference for the data collected during the test. The start of a test may be indicated by an onset of acceleration.

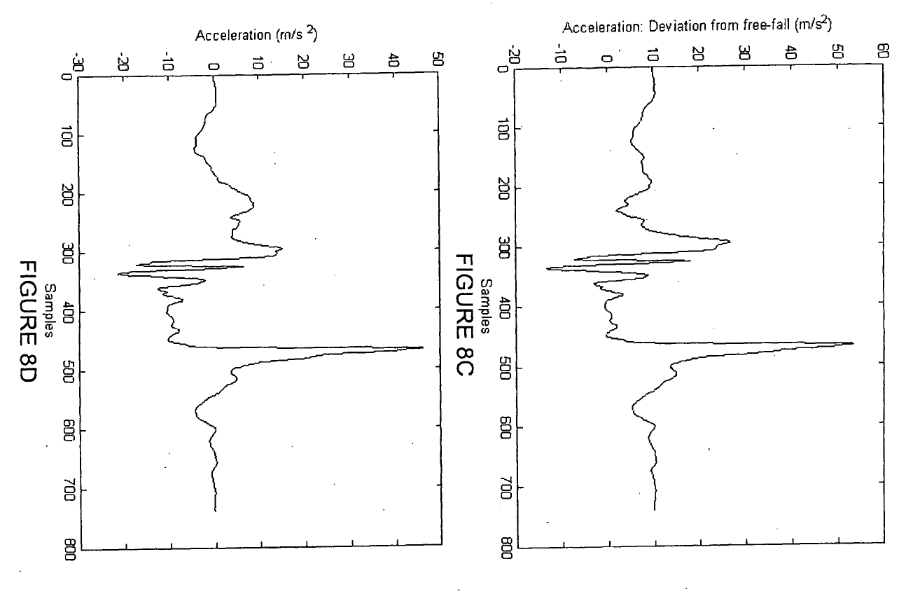

The following description uses examples of a jump test and a running test for illustrative purposes, but it is to be understood that other types of tests may also be conducted according to certain embodiments of the invention. FIG. 8A shows example Z-axis acceleration data from a jump test which is used to illustrate the operation of method 100 in the following paragraphs.

At block 104 the processor determines if all data for the test has been received. The processor may determine if all data for the test has been received by comparing the received data with an expected data pattern and/or checking timing information which may be included in the data. If all data for the test has not been received (block 104 NO output), the processor requests the missing data at block 106 and the steps of blocks 102 and 104 are repeated.

When all data for the test has been received (block 104 YES output), the processor applies a scaling function to the data at block 108. At block 110 the processor corrects the data for sensor gain and bias. Sensor gain an bias may be determined prior to the initiation of method 100 by calibrating the sensors used to collect the data. FIG. 8B shows the example jump test Z-axis acceleration data of FIG. 8A after the scaling function has been applied and the data has been corrected for gain and bias.

At block 112 the processor crops the data by detecting the data corresponding to the stationary periods before and after the test, and discarding data collected before and after the first and second stationary periods, respectively. FIG. 8C shows the example Z-axis acceleration data after cropping.

At block 114 the processor determines an orientation of the sensors used to collect the acceleration data based on the rotation data. The processor then applies a rotation function to the acceleration data based on the determined orientation to produce acceleration data along three global axes. The global axes may comprise, for example, a vertical axis, a lateral axis and a longitudinal axis. The processor then subtracts g (the acceleration due to gravity) from the acceleration data along the vertical axis to produce global acceleration data. In the jump test, the vertical axis may be referred to as the primary axis since vertical acceleration data is primarily used to extract features relating to athletic performance. FIG. 8D shows the global vertical acceleration data produced from the example acceleration data after the steps of block 114.

At block 116 the processor applies boundary conditions to the global acceleration data. For example, the processor may require the global acceleration data to indicate zero acceleration over the stationary periods and adjust all of the global acceleration data so that zero acceleration is indicated for the stationary periods. FIG. 8E shows the global vertical acceleration data after the steps of block 116.

At block 118 the processor processes the global acceleration data. For example, at block 118 the processor may integrate the global acceleration data to produce global velocity data. The integration performed by the processor may be, for example, a numerical integration using the trapezoidal rule. FIG. 8F shows the global velocity data produced from the global acceleration data of FIG. 8E. Other examples of processing performed at block 118 include filtering the global acceleration data and differentiating the global acceleration data. Filtration and/or differentiation of the global acceleration data may be performed instead of or in combination with integration of the global acceleration data.

At block 120 the processor extracts features relating to athletic performance from the processed data. The processor extracts the features based on a test identification which may be specified by a user. The processor may extract the features by detecting a plurality of expected events in the data, as described further below.

At block 121 the processor outputs the extracted features. The extracted features may be output, for example, by displaying one or more graphs (e.g., acceleration, force, power, velocity, and/or position versus time) or values (e.g., reaction time, preload time, maximum force, etc.) on a display, as described further below.

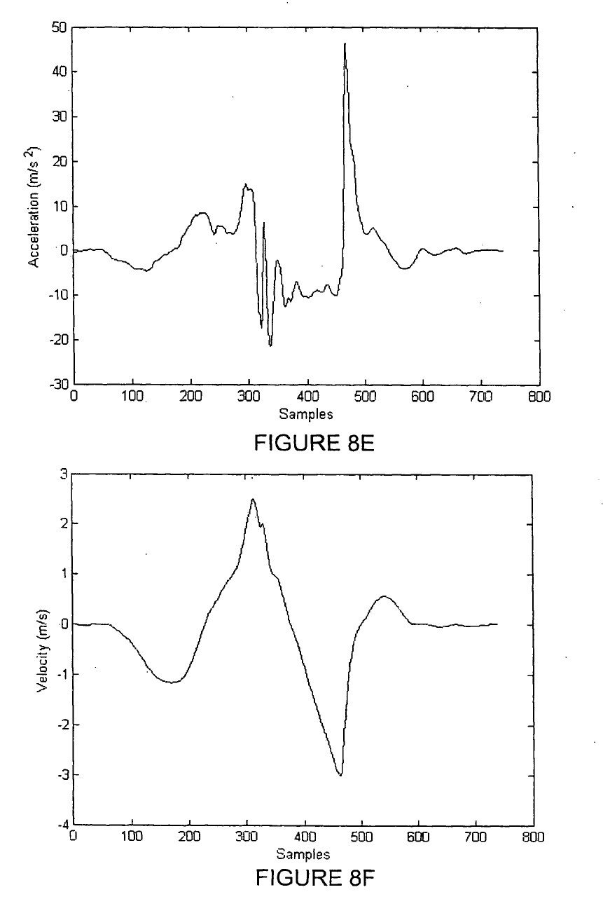

FIG. 9 is a flowchart illustrating one possible method of extracting features in block 120 of FIG. 7. At block 122 the processor receives global acceleration and velocity data. At block 124 the processor receives a test identification which specifies the type of test which was performed to produce the global acceleration and velocity data. The test identification may include a plurality of expected events. As indicated by the dashed box around blocks 122 and 124, the order of these steps is not important.

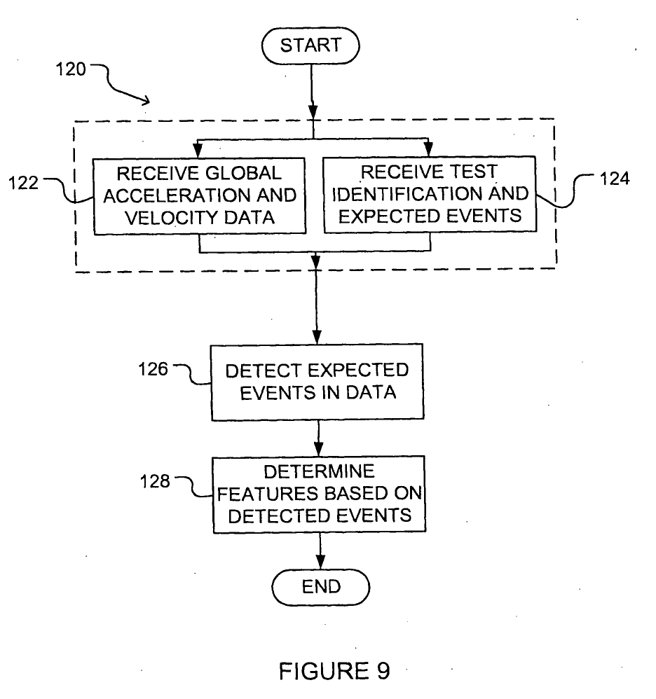

At block 126 the processor detects events in the global acceleration data which correspond to the expected events. FIG. 10 illustrates some detected events in the example jump test vertical acceleration data of FIGS. 8E and 8F. Event 130 corresponds to the initiation of a jumping motion by an athlete flexing their legs and moving their torso downwardly, and is characterized by the beginning of a negative vertical acceleration. Event 132 corresponds to the beginning of the athlete's upward push, and is characterized by a transition from a negative to a positive acceleration. Event 134 corresponds to the point at which the athlete increases the development of force, and is characterized by an increase in positive vertical acceleration. Event 136 corresponds to the point at which the athlete's toes leave the ground, and is characterized by a fast transition from a positive acceleration to a negative acceleration. Event 138 corresponds to the point at which the athlete's feet initially impact the ground, and is characterized by a fast transition from a negative acceleration to a large positive acceleration. Event 140 corresponds to the end of the “impact phase”, and is characterized by a transition from positive to negative acceleration. The time between two events may be determined from the number of samples between these events and the sampling rate. Although the illustrated example uses vertical acceleration data, it is to be understood that global acceleration data for other axes, as well as rotation data, may also be analyzed to detect expected events.

At block 128 the processor determine features relating to athletic performance based on the detected events. Features which may be determined for a jump test include:
Reaction Time—the time between when an audible signal is sounded to start the test and when the athlete begins to move;
Jump Start—where the athlete begins moving down;
Preload Time—the time the athlete takes to bend down;
Start of Upwards Motion—where the athlete begins moving upwards;
Push-off Time—the time the athlete takes to push and reach toe-off;
Take-off Velocity—upward velocity at toe-off;
Toe-off—where the athlete leaves the ground;
Air Time—the time the athlete is in the air;
Height Jumped—height that the athlete jumps;
   (This feature may be determined based on either Air Time or Take-off Velocity, or both, to provide for data verification. If the two determinations differ by more than a predetermined amount, an error signal may be generated.)
Maximum Take-off Force—the maximum force generated in the take-off phase (between “start of upwards motion” and “toe-off”);
Mean Take-off Force—the average amount of force generated in the take-off phase;
Maximum Take-off Power—the maximum power generated in the take-off phase;
Mean Take-off Power—the average amount of power generated in the take-off phase;
Maximum Rate of Force Development—the maximum rate that force is developed in the take-off phase;
Mean Rate of Force Development—the average rate of force developed in the take-off phase;
Ground Contact—where the athlete contacts the ground;
End of Impact—the time from landing until athlete completes landing and stops
Impact Time—time between “Ground Contact” and “End of Impact”;
Maximum Impact Force—the amount of force the athlete creates upon landing; and,
Mean Impact Force—the average amount of force in the landing phase (between “ground contact” and “end of impact”).
   Methods and systems according to the invention may also be used to extract features from data from a multiple test or a squatting jump test. In a multiple jump test, the athlete performs a series of jumps. The above features may be extracted from data from a each jump of a multiple jump test, in addition to features such as the ability of the athlete to maintain a particular jump height, and the amount of force and power the athlete can repeatedly produce. In a squatting jump test, the athlete begins from a squatting position, and all of the above features may be extracted from squatting jump test data except for “Preload Time”, since the athlete begins in the squatting position.

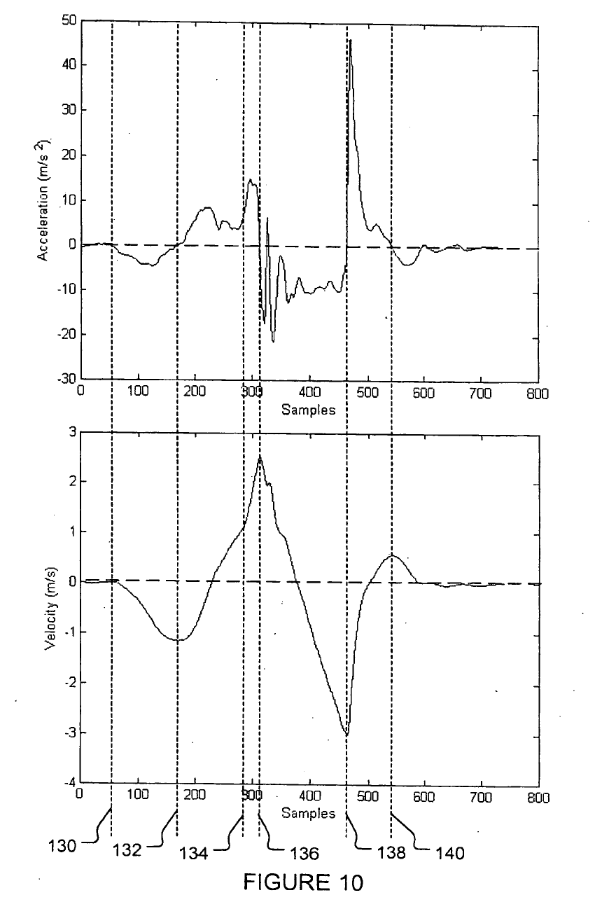

Methods and systems according to the invention may also be used to extract features relating to athletic performance from tests other than jump tests. For example, FIG. 11 shows example acceleration and rotation data from a running test. In a run test, the primary axis may be the longitudinal axis positioned along the forward, and events corresponding to expected events may be detected in the forward acceleration data to extract features. Features which may be extracted from data collected during a running test include:
Reaction Time—the time between when an audible signal is sounded to start the test and when the athlete begins to move;
Number of steps—number of times a foot leaves the ground;
Step Length—the length of each step from when one foot touches the ground to when the other foot touches the ground;
Stride Length—the length of each stride from when one foot touches the ground to when the same foot touches the ground again (one stride equals two steps);
Stride Rate—frequency of stride;
Toe Offs—where each foot leaves the ground;
Initial Contacts—where each foot strikes the ground;
Air Time—time athlete is not touching the ground between each step;
   (A high air time corresponds with a fast athlete.)
Ground Contact Time—time the athlete is touching the ground between each step;
   (A high ground contact time corresponds with a slow athlete.)
Total Air Time—total time the athlete is not touching the ground in an entire running test;
Total Ground Contact Time—total time the athlete is touching the ground in an entire running test;
Acceleration Efficiency—a measure of acceleration in one direction versus accelerations in other directions;
   (Acceleration efficiency may be calculated by, for each direction (forward, backward, left, right, up, down) taking a sum of all of the positive accelerations in that direction, and dividing by the sum of all positive accelerations in all of the six directions. The goal for runners is generally to minimize all accelerations except for forward accelerations to give maximal speed with minimum wasted energy.)
Power Efficiency—a measure of forward power versus power in other directions (backward, left, right, up, down);
   (Power efficiency may be calculated in a manner similar to acceleration efficiency. Sprinters aim to maximize the power in the forward direction while minimizing all other powers.)
Roll—the amount of rotation about the Y-axis (bending at the hips);
   (Sprinters aim to minimize Roll.)
Yaw—the amount of rotation about the Z-axis (turning of the hips);
   (Sprinters aim to minimize Yaw.)
Left/Right symmetry—amount of acceleration left and right;
   (Sprinters aim to minimize left/right accelerations and any differences between left and right accelerations.)
Time to top 90%—the time it takes an athlete to reach 90% of their peak velocity; and,
Velocity Maintenance—how long the athlete can remain within 90% of their peak velocity.

Methods and systems according to the invention may be used to extract features from data collected during any type of test. In each case, a set of events that are expected to occur in the acceleration and/or rotation data are stored in a memory accessible by a processor programmed to extract features relating to athletic performance, such as feature extractor 32 of FIG. 2. The processor detects events in the acceleration and/or rotation data which correspond to the expected events for the selected test, and extracts features based on characteristics of the detected events such as the time the events occur, the acceleration, velocity, position, and power generated at the time of the events, integrations of acceleration and/or rotation data between events, and the like.

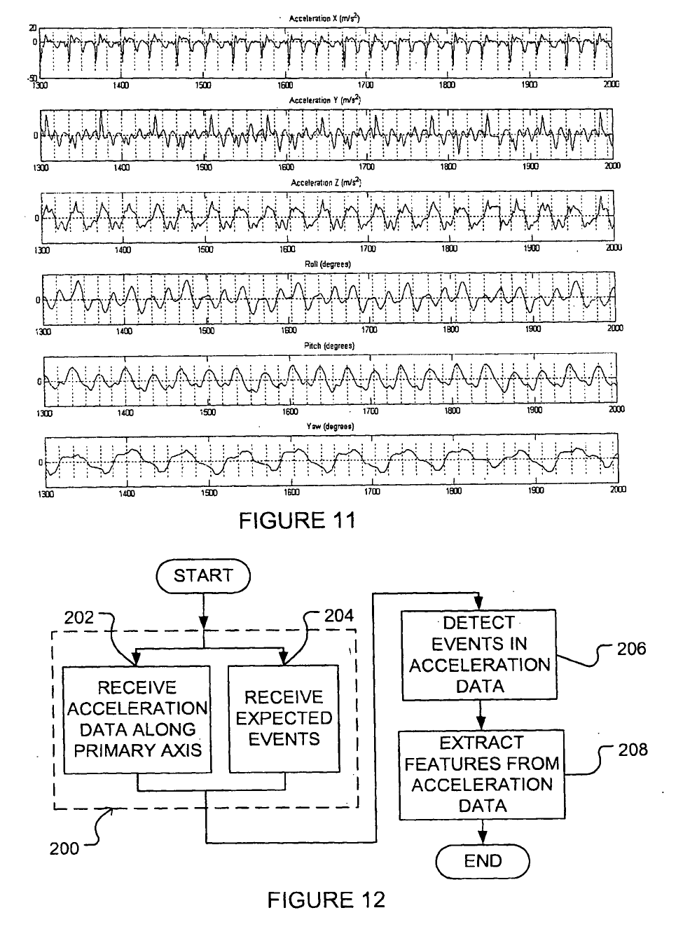

FIG. 12 is a flowchart illustrating a method 200 for assessing athletic performance according to another embodiment of the invention. Method 200 may be carried out, for example, by a suitable processor. At block 202, the processor receives data representing acceleration along a primary axis. For a jump test, the primary axis is the vertical axis. For a running test, the primary axis is the longitudinal (i.e. forward/backward) axis. At block 204 the processor receives information specifying a plurality of expected test events. As indicated by the dashed box around blocks 202 and 204, the order of these steps is not important.

At block 206 the processor detects events in the acceleration data which correspond to the expected test events. At block 208 the processor extracts features relating to athletic performance from the acceleration data based on the detected events.

In operation, an athlete attaches a sensing device to their body, for example, by putting on a belt which holds the sensing device at the small of their back. The athlete's trainer or coach turns on the base unit and selects one of a plurality of predetermined tests using an interactive display or other input/output device and informs the athlete to prepare to begin the selected test. The base unit sends a test initiation signal to the sensing device, which in turn provides the athlete with a start signal. The athlete then performs the test, and the sensing device collects data during the test and provides the collected data to the base unit.

The base unit extracts features relating to athletic performance by detecting events in the data which correspond to expected events for the selected test. The base unit outputs the extracted features to the coach or trainer by means of the input/output device. The extracted features may be outputted after the test has been completed, or in real time during the test. In embodiments where the extracted features are outputted in real time, the coach or trainer may provide the athlete with feedback based on the extracted features in order to improve the athlete's performance.

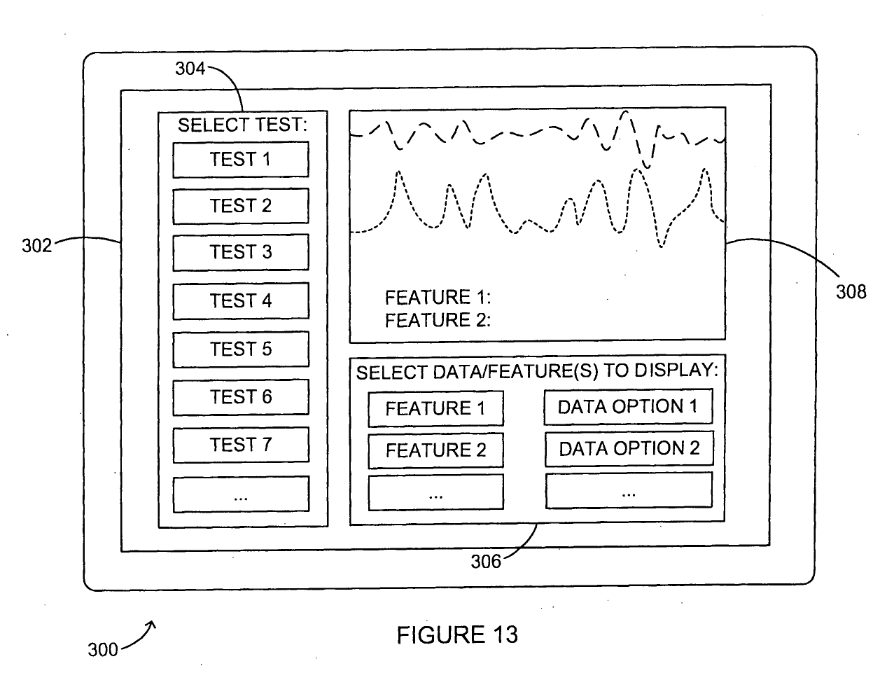

FIG. 13 illustrates an example input/output device 300 according to one embodiment of the invention. Input/output device 300 comprises a touch-sensitive display screen 302. Screen 302 may be driven by a processor to display a test selection area 304 which lists a plurality of predetermined tests which a user may select by pressing screen 302 at the location where the name of the desired test is displayed. Screen 302 may also be driven to display a data/feature selection area 306 which lists a plurality features and data display options which a user may select by pressing screen 302 at the location where the desired feature/data option is displayed. Screen 302 may display the selected features and data options in a display area 308.

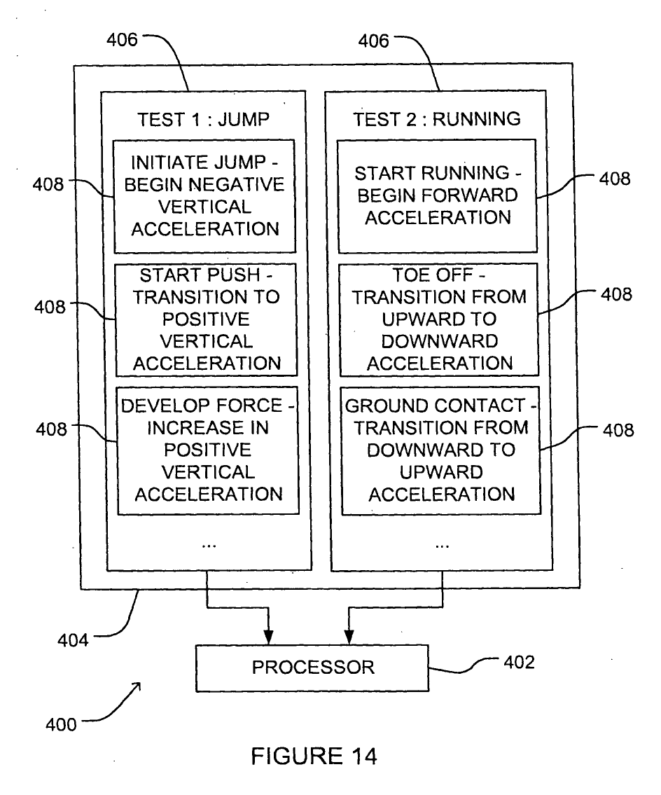

FIG. 14 shows an example feature extractor 400 according to one embodiment of the invention. Feature extractor 400 comprises a processor 402 coupled to a memory 404. A plurality of test identifications 406 are stored in memory 404. Each test identification 406 includes a plurality of expected events 408. In the illustrated example, a jump test and a running test are shown with some of their respective events, as discussed above, but it is to be understood that memory 404 could have additional test identifications 406 stored therein.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

What is claimed is:

1. A system for assessing athletic performance, the system comprising:
a mounting device wearable by an athlete;
a sensing device attachable to the mounting device, the sensing device comprising:
a plurality of acceleration sensors for measuring acceleration data along three local axes during an athletic test to produce at least three acceleration signals;
a plurality of rotation sensors for measuring rotation data about said three local axes during the athletic test to produce at least three rotation signals;
signal conditioning hardware for conditioning the acceleration and rotation signals and sampling the acceleration and rotation signals at a sampling rate to produce acceleration and rotation data; and,
a wireless communication device for transmitting the acceleration and rotation data; and,
a base unit comprising:
a wireless communication device for receiving the acceleration and rotation data from the sensing device;
a feature extractor for extracting features relating to athletic performance from the acceleration and rotation data based on a plurality of expected events of the athletic test; and,
an output device for outputting the features relating to athletic performance.

2. A system according to claim 1 wherein the mounting device comprises a strap configured to fit around the athlete's waist such that the sensing device rests in the small of the athlete's back.

3. A system according to claim 1 wherein the sensing device comprises at least one temperature sensor for measuring temperature of the acceleration and rotation sensors and producing a temperature signal.

4. A system according to claim 1 wherein the plurality of acceleration sensors comprise at least two accelerometers associated with each of the three local axes.

5. A system according to claim 4 wherein the at least two accelerometers associated with each of the three local axes comprise a high range accelerometer and a high sensitivity accelerometer.

6. A system according to claim 1 wherein the sensing device comprises a plurality of magnetometers for measuring the earth's magnetic field and producing a magnetic heading signal.

7. A system according to claim 1 wherein the sensing device comprises a pressure sensor for measuring an atmospheric pressure and producing a pressure signal.

8. A system according to claim 1 wherein the base unit comprises an input device for receiving the test identification.

9. A system according to claim 1 wherein the sensing device comprises signal processing means for collecting the acceleration and rotation data.

10. A system according to claim 1 wherein the sensing device comprises an audio device for indicating a beginning of a test period to the athlete.

11. A method for assessing athletic performance of a living subject, the method comprising:
providing at least three acceleration sensors on the subject configured to measure acceleration along three local axes;
providing at least three rotation sensors on the subject configured to measure rotation about said three local axes;
monitoring the acceleration sensors and the rotation sensors to produce acceleration data and rotation data;
determining an orientation of said three local axes based on the measured rotation data;
applying a rotation function to the measured acceleration data based on the determined orientation of said three local axes to generate corrected acceleration data along three global axes;
receiving a test identification specifying a plurality of expected events;
extracting features relating to athletic performance of the subject by detecting events corresponding to the expected events in the corrected acceleration data; and
outputting the extracted features.

12. A method according to claim 11 wherein the test identification identifies a jump test and the plurality of expected events comprises an initiation of a jumping motion characterized by an onset of negative vertical acceleration.

13. A method according to claim 12 wherein the plurality of expected events comprises a start of an upward push characterized by a transition from negative to positive vertical acceleration.

14. A method according to claim 13 wherein extracting features comprises determining a preload time between the initiation of the jumping motion and the start of the upward push.

15. A method according to claim 13 wherein the plurality of expected events comprises a toe-off characterized by a fast transition from positive to negative vertical acceleration.

16. A method according to claim 15 wherein extracting features comprises determining a maximum force applied between the start of the upward push and the toe-off.

17. A method according to claim 15 wherein extracting features comprises determining an average force applied between the start of the upward push and the toe-off.

18. A method according to claim 15 wherein extracting features comprises determining a take-off velocity.

19. A method according to claim 15 wherein the plurality of expected events comprises a ground contact characterized by a fast transition from negative to positive vertical acceleration.

20. A method according to claim 19 wherein the plurality of expected events comprises an end of ground impact characterized by a transition from positive to negative vertical acceleration.

21. A method according to claim 11 wherein the test identification identifies a running test and the plurality of expected events comprises a plurality of initial contacts, each initial contacts characterized by a fast transition from negative to positive vertical acceleration.

22. A method according to claim 21 wherein the plurality of expected events comprises a plurality of toe-offs, each toe-off characterized by a transition from positive to negative vertical acceleration.

23. A method according to claim 22 wherein extracting features comprises determining a total air time for the running test.

24. A method according to claim 22 wherein extracting features comprises determining a total ground contact time for the running test.

25. A method for assessing athletic performance, the method comprising:
providing at least one acceleration sensor for measuring acceleration along a primary axis;
monitoring the acceleration sensor during a test period to produce acceleration data;
receiving information specifying a plurality of expected test events;
detecting events in the acceleration data corresponding to the expected test events based on the information received; and,
extracting features relating to athletic performance from the acceleration data based on the detected events.

26. A system for assessing athletic performance, the system comprising:
at least one acceleration sensor attachable to an athlete for measuring acceleration data along a primary axis during an athletic test to produce at least one acceleration signal;
a processor for receiving the acceleration signal and sampling the acceleration signal at a sampling rate to produce acceleration data;
a feature extractor for extracting features relating to athletic performance from the acceleration and rotation data based on a plurality of expected events of the athletic test; and,
an output device for outputting the features relating to athletic performance.
